(19) United States
(12) Patent Application Publication
(10) Pub. No.: US 2026/0209251 A1
(43) Pub. Date: Jul. 23, 2026

(54) XANTHENE COMPOUNDS HAVING A WAVELENGTH IN THE RANGE FROM 650 TO 1200 NM

(71) Applicant: BASF SE, Ludwigshafen am Rhein (DE)

(72) Inventors: Hans REICHERT, Basel (CH); Christian DOERR, Ludwigshafen am Rhein (DE)

(21) Appl. No.: 19/141,303

(22) PCT Filed: Dec. 13, 2023
(86) PCT No.: PCT/EP2023/085672

§ 371 Date: Jun. 19, 2025

(30) Foreign Application Priority Data
Dec. 21, 2022 (EP) 22215528.5

(51) Int. Cl. C07D 519/00; A61K 49/00; C09D 11/037; G01N 21/359

(52) U.S. Cl. CPC C07D 519/00; A61K 49/0041; C09D 11/037; G01N 21/359

(57) ABSTRACT

The present invention relates to compounds of the formula

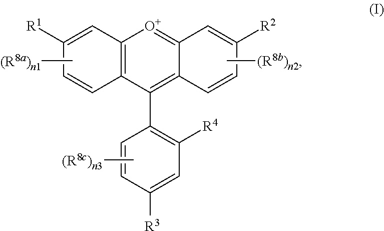

a process for its preparation and its use as compound that absorbs light emitted from an irradiation source and optionally emits light different from that of the irradiation source and having a wavelength in the range from 650 to 1200 nm; in photovoltaic applications; or in bioimaging, or in photodynamic therapy; or as semiconductor in organic electronic applications; as laser dye, in an ink for machine readability and/or security applications or for the laser-welding of plastics; or for brand protection or as marker for liquids. The compounds of formula (I) may have a high molar extinction coefficient, a high fluorescence quantum yield, a high solubility and stability in the application medium, good storage stability and/or good detectability even in very small amounts in correspondingly marked liquids.

The present invention relates to compounds of the formula

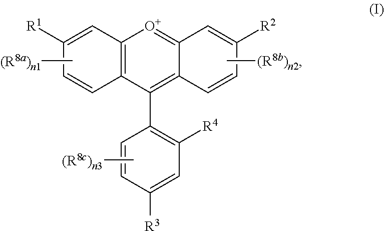

a process for its preparation and its use as compound that absorbs light emitted from an irradiation source and optionally emits light different from that of the irradiation source and having a wavelength in the range from 650 to 1200 nm; in photovoltaic applications; or in bioimaging, or in photodynamic therapy, or as semiconductor in organic electronic applications; as laser dye, in an ink for machine readability and/or security applications or for the laser-welding of plastics; or for brand protection or as marker for liquids. The compounds of formula (I) may have a high molar extinction coefficient, a high fluorescence quantum yield, a high solubility and stability in the application medium, good storage stability and/or good detectability even in very small amounts in correspondingly marked liquids.

### DESCRIPTION OF THE RELATED ART

WO2009/094536A1 discloses compounds of Formula III

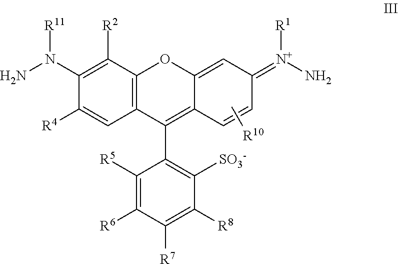

or a stereoisomer, tautomer, hydrate, solvate, or salt thereof; wherein R¹ is selected from the group consisting of H, alkyl and substituted alkyl; R², R⁴, R⁵, R⁶, R⁷, R⁸ and R¹⁰ are each independently selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃′, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl; and R¹¹ is selected from the group consisting of H, alkyl, substituted alkyl, aryl, substituted aryl, heteroaryl, substituted heteroaryl, heterocyclyl, substituted heterocyclyl, cycloalkyl, and substituted cycloalkyl; and its use for detecting analytes containing aldehyde and ketone groups.

US20220056335A1 relates to near infrared dyes comprising a structure of Formula I,

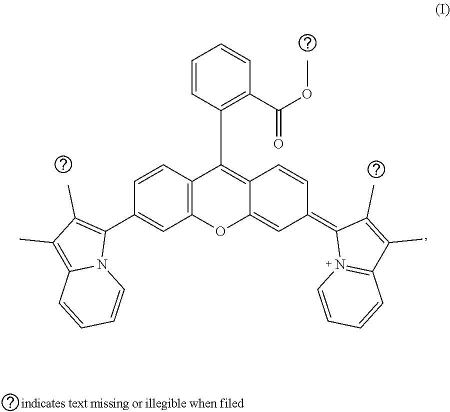

wherein R¹ comprises a substituted or unsubstituted C₁-C₁₀ linear or branched alkyl group, a substituted or unsubstituted C₁-C₁₀ linear or branched alkoxy group, a C₃-C₁₀ cycloalkyl group, a substituted or unsubstituted aryl or heteroaryl group, or any combination thereof; and wherein R² comprises a C₁-C₁₀ linear or branched alkyl group, a C₃-C₁₀ cycloalkyl group, or any combination thereof, methods of making same, compositions comprising same and methods of using the compositions to perform imaging on biological samples, and optoelectronic devices using the dyes.

Chathuranga Rathnamalala et al., J. Org. Chem. 2019, 84, 13186-13193 report the synthesis of a NIR II emissive dye by the C—H bond functionalization of 1-methyl-2-phenylindolizine with 3,6-dibromoxanthene. The rhodindolizine (RhIndz) spirolactone product was nonfluorescent; however, upon opening of the lactone ring by the formation of the ethyl ester derivative,

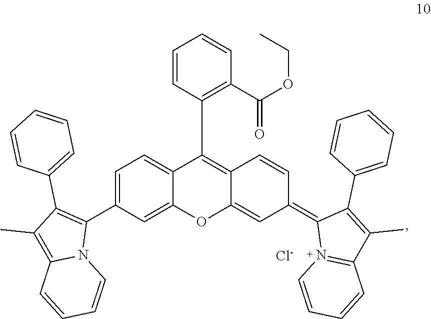

the fluorophore absorbs at 920 nm and emits at 1092 nm, which are both in the NIR II region.

Satadru Chatterjee et al., RSC Adv., 2021, 11, 27832 report the synthesis and characterization of two Shortwave infrared (SWIR) emissive xanthene-based RosIndolizine dyes coded ^{Ph}RosIndz and ^{tol}RosIndz,

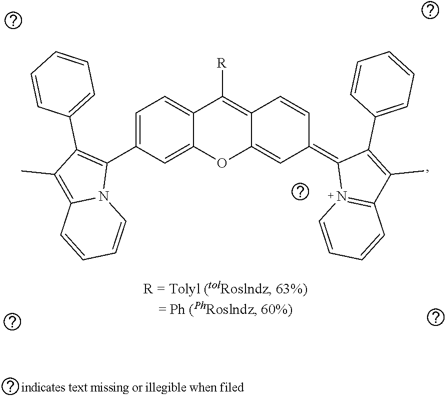

which have great potential for deep-tissue in vivo biological imaging with high resolution. The emission of these dyes is shown in the SWIR region with peak emission at 1097 nm.

Satadru Chatterjee et al., J. Org. Chem. 2022, 87, 11319-11328 report the synthesis of four novel rhodindolizine dyes, such as, for example,

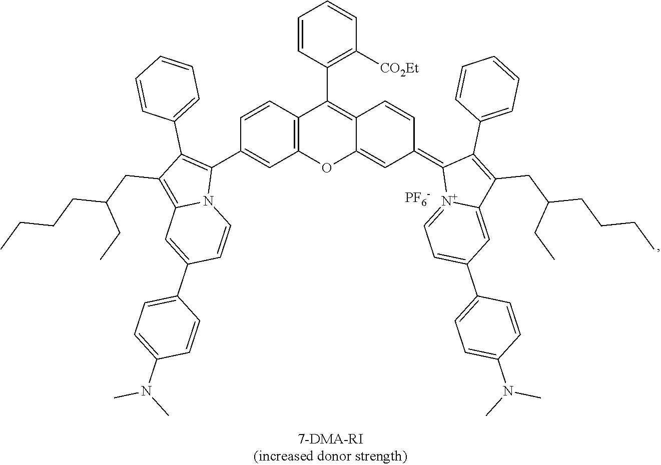

with varied steric elements at the xanthene core, electronics at the indolizine donors, and functionality for click reactions on the dyes. These dyes were characterized photophysically via steady-state absorption and emission spectroscopy to show appreciable SWIR absorption (λₘₐₓ^{abs}=926-1096 nm with onsets all >1000 nm) and emission (λₘₐₓ^{em}=1082-1256 nm).

WO00/064988 relates to compounds of formula

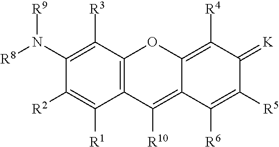

R¹ and R⁶ are H;
R², R³, R⁴ and R⁶ are independently H, F, Cl, Br, I, CN; or C₁-C₁₈ alkyl, or C₁-C₁₆ alkoxy, where each alkyl or alkoxy is optionally further substituted by F, Cl, Br, I, a carboxylic acid, a salt of carboxylic acid, or a carboxylic acid ester of a C₁-C₆ alcohol; or —SO₃X where X is H or a counterion;
or R¹ taken in combination with R², or R⁶ taken in combination with R⁶ is a fused six-membered aromatic ring;
R⁸ and R⁹ are independently H, C₁-C₆ alkyl, C₁-C₆ carboxyalkyl, C₁-C₆ sulfoalkyl, a salt of C₁-C₆ carboxyalkyl, or a salt of C₁-C₆ sulfoalkyl, wherein the alkyl portions are optionally substituted by amino, hydroxy, carboxylic acid, a salt of carboxylic acid, or a carboxylic acid ester of a C₁-C₆ alkyl; or -L-R_{X}; or -L-S_{C}; or one or more of R⁸ and R⁹ is a Q moiety; or
R⁸ taken in combination with R⁹ forms a saturated 6- or 6-membered heterocycle that is optionally fused to a Q moiety and optionally further substituted by methyl, carboxylic acid, a salt of carboxylic acid, a carboxylic acid ester of a C₁-C₆ alkyl, or by -L-R_{X}, or -L-S_{C}; wherein each Q moiety is 1-4 aromatic or heteroaromatic rings, which rings are fused to each other when there are 2-4 of them and, irrespective of number, are optionally substituted by halogen, cyano, sulfo, alkali or ammonium salt of sulfa, carboxy, alkali or ammonium salt of carboxy, nitro, alkyl, perfluoroalkyl, alkoxy, alkylthio, amino, monoalkylamino, dialkylamino; alkylamido; or is substituted by -L-R_{X}; or is substituted by -L-S_{C}; wherein each heteroaromatic ring in Q is a 5- or 6-membered aromatic heterocycle having 1 to 3 heteroatoms selected from the group consisting of O, N or S in any combination; and
K is O or N⁺R¹⁸R¹⁹;
wherein R¹⁸ and R¹⁹ are independently H, C₁-C₆ alkyl, C₁-C₆ carboxyalkyl, C₁-C₆ sulfoalkyl, a salt of C₁-C₆ carboxyalkyl, or a salt of C₁-C₆ sulfoalkyl, wherein the alkyl portions are optionally substituted by amino, hydroxy, carboxylic acid, a salt of carboxylic acid, or a carboxylic acid ester of a C₁-C₆ alkyl; or -L-R_{X}; or -L-S_{C}; or one or more of R¹⁸ and R¹⁹ is a Q moiety; or R¹⁸ taken in combination with R¹⁹ forms a saturated 5- or 6-membered heterocycle that is a piperidine, or a pyrrolidine that is optionally fused to a Q moiety, and optionally further substituted by methyl, carboxylic acid, a salt of carboxylic acid, a carboxylic acid ester of a C₁-C₆ alkyl; or -L-R_{X}; or -L-S_{C};
R¹⁰ is H, CN, a carboxylic acid, a salt of carboxylic acid, or a carboxylic acid ester of a Cr Cc alcohol; or R¹⁰ is a saturated or unsaturated C₁-C₁₈ alkyl that is optionally substituted one or more times by F, Cl, Br, carboxylic acid, a salt of carboxylic acid, a carboxylic acid ester of a C₁-C₆ alcohol, —SO₃X, amino, alkylamino, or dialkylamino, the alkyl groups of which have 1-6 carbons; or R¹⁰ has the formula where R¹², R¹³, R¹⁴, R¹⁵ and R¹⁶ are independently H, F, Cl, Br, I, —SO₃X, a carboxylic acid, a salt of carboxylic acid, CN, hydroxy, amino, hydrazino; or C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions of which are optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino or alkoxy, the alkyl portions of each having 1-6 carbons; or one pair of adjacent substituents R¹³ and R¹⁴, R¹⁴ and R¹⁵ or R¹⁵ and R¹⁶, when taken in combination, form a fused 6-membered aromatic ring that is optionally further substituted by carboxylic acid, or a salt of carboxylic acid; or one of R¹², R¹³, R¹⁴, R¹⁵ and R¹⁶ is -L-R_{X} or -L-S_{C}; provided that at least one of R⁵, R⁹, R¹⁸, and R¹⁹ is, or is fused to, a Q moiety; and further provided that at least one of R⁸, R⁹, R¹², R¹³, R¹⁴, R¹⁵, R¹⁶, R¹⁸, or R¹⁹ is -L-R_{X} or -L-S_{C}; or at least one Q moiety is substituted by -L-R_{X} or -L-S_{C}; wherein
L is a covalent linkage; and
R_{X} is a reactive functional group that is a maleimide, isocyanate, isothiocyanate, a phosphoramidite, a reactive platinum complex, perfluorobenzamido, azidoperfluorobenzamido, a succinimidyl ester, a sulfosuccinimidyl ester, an alkali or alkaline earth metal salt of a sulfosuccinimidyl ester, a symmetric anhydride, a mixed anhydride of a chloroformate having 2-8 carbons, a mixed anhydride of a carboxylic acid or perfluorinated carboxylic acid having 2-8 carbons, a mixed anhydride of a sulfonic acid or fluorinated sulfonic acid having 1-8 carbons, or an ester of a phenol or a naphthol that is further substituted one or more times by nitro, sulfo, carboxy, alkali or alkaline earth metal salt of sulfo or carboxy, cyano, fluoro, chloro, or trifluoromethyl; or R_{X} is the adduct of a carboxylic acid and a carbodiimide having 2-14 carbons; and
S_{C} is a conjugated substance, such as, for example, a compound of formula

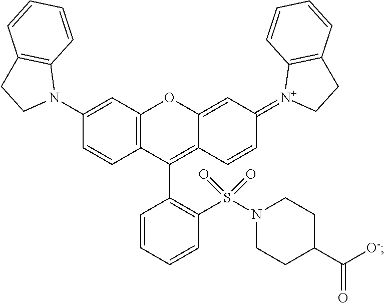

and their application as luminescence quenching compounds.

In the field of fuel marking, interfering influences of the fuel matrix can be minimized by using NIR II absorbing and emitting markers. There is an unmatched need for chromophores that absorb and emit especially in the NIR region, very especially NIR II region. In the field of fluorescence biological imaging applications, the availability of NIR emissive molecules stays an important need.

Thus, it is an object of the present invention to provide compounds that may have a high stability, high absorption coefficient and/or fluorescence quantum yield compared to compounds known in the art, especially in the NIR region, very especially NIR II region.

### SUMMARY OF THE INVENTION

It was surprisingly found that these and further objectives are achieved by the compounds of formula (I) as defined herein below.

Accordingly, the present invention relates to compounds of the formula

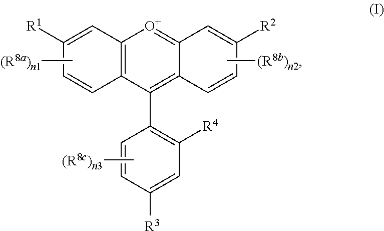

wherein
R¹ and R² are independently of each other a group of formula

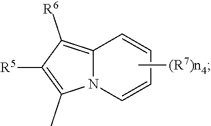

R³ is hydrogen, —CO₂H, or —SO₃H;
R⁴ is —SO₃⁻, or a group of formula —SO₂NR¹²R¹³, with the proviso that in case R⁴ is not negatively charged, the compound of the formula (I) comprises an anionic counterion; R⁵ is a C₁-C₂₄alkyl group, a C₃-C₁₀-cycloalkyl group, substituted with one or more substituents R¹⁴, a C₆-C₁₀aryl-C₁-C₁₀alkylene, wherein may be substituted with one or more substituents R¹⁴ and the alkylene group may be interrupted by one or more nonadjacent groups selected from O, S and —NR⁹; a C₆-C₁₀aryl group, which may be substituted with one or more substituents R¹⁴, or a C₂-C₁₄heteroaryl group, which may be substituted with one or more substituents R¹⁴;
R⁶ is hydrogen, a C₁-C₂₄alkyl group, a C₁-C₂₄alkoxy group, a C₆-C₁₀-aryl group, which may be substituted with one or more substituents R¹⁴, and a C₁-C₂₄-fluoroalkyl group, fluorine, chlorine or bromine;
R⁷ is a C₁-C₂₄alkyl group, a C₁-C₂₄alkoxy group, a C₁-C₂₄-fluoroalkyl group, a group NR¹⁰R¹¹, fluorine, chlorine or bromine;
R^{8a}, R^{8b} and R^{8c}, independently of each other, are selected from a C₁-C₂₄alkyl group, a C₁-C₂₄alkoxy group, a C₁-C₂₄-fluoroalkyl group, fluorine, chlorine or bromine;
R⁹ is hydrogen, or a C₁-C₄alkyl group;
R¹⁰ and R¹¹ independently of each other a C₁-C₂₄alkyl group, a hydroxy-C₁-C₂₄alkyl group, a C₆-C₁₀-aryl group, which may be substituted with one or more substituents R¹⁴, or a C₃-C₁₀-cycloalkyl group, substituted with one or more substituents R¹⁴; or
R¹⁰ and R¹¹ together with the nitrogen to which they are bonded form a five, or six membered ring system, which may be interrupted by —O—, —S—, or —NR¹²′— and may be part of a fused ring system, which may be substituted with one or more substituents R¹⁴;
R¹² and R¹³ are independently of each other hydrogen, a C₁-C₂₄alkyl group, a hydroxy-C₁-C₂₄alkyl group, a C₃-C₁₀-cycloalkyl group, substituted with one or more substituents R¹⁴, a C₆-C₁₀aryl-C₁-C₁₀alkylene, wherein may be substituted with one or more substituents R¹⁴ and the alkylene group may be interrupted by one or more nonadjacent groups selected from O, S and —NR¹²′; a C₆-C₁₀aryl group, which may be substituted with one or more substituents R¹⁴, or a C₂-C₁₄heteroaryl group, which may be substituted with one or more substituents R¹⁴; or
R¹² and R¹³ together with the nitrogen to which they are bonded form a five, or six membered ring system, which may be interrupted by —O—, —S—, or —NR¹²′— and may be part of a fused ring system, which may be substituted with one or more substituents R^{14′};
R¹²′ is hydrogen, or a C₁-C₄alkyl group;
R¹⁴ is a C₁-C₁₀alkyl group, a C₁-C₁₀alkoxy group, a C₁-C₁₀fluoroalkyl group, a nitro group, a cyano group, a hydroxy group, fluorine, chlorine or bromine;
R^{14′} is a C₁-C₁₀alkyl group, a C₁-C₁₀alkoxy group, a C₁-C₁₀fluoroalkyl group, a nitro group, a cyano group, a hydroxy group, a —(CH₂)ₙ₅COOH group, a —(CH₂)ₙ₅COO⁻ group, fluorine, chlorine or bromine;
and n1 is 0, 1, or 2; n2 is 0, 1, or 2, n3 is 0, or 1, n4 is 0, 1, or 2 and n5 is 0, or an integer i to 10.

A further aspect of the present invention relates to the use of the compound of formula (I) as defined above as fluorescent dye that absorbs light emitted from an irradiation source and optionally emits light different from that of the irradiation source and having a wavelength in the range from 650 to 1200 nm, especially 680 to 1200 nm.

A further aspect of the present invention relates to the use of the compound of formula (I) as defined above as fluorescent dye in a converted LED.

A further aspect of the present invention relates to the use of the compound of formula (I) as defined above in a near infrared spectrometer apparatus.

A further aspect of the present invention relates to the use of the compound of formula (I) as defined above in an agricultural film.

A further aspect of the present invention relates to the use of the compound of formula (I) as defined above in photovoltaic applications.

A further aspect of the present invention relates to the use of the compound of formula (I) as defined above in a fluorescent solar concentrator.

A further aspect of the present invention relates to the use of the compound of formula (I) as defined above as semiconductor in organic electronic applications.

A further aspect of the present invention relates to the use of the compound of formula (I) as defined above as laser dye, in an ink for machine readability and/or security applications or for the laser-welding of plastics.

A further aspect of the present invention relates to the use of the compound of formula (I) as defined above for brand protection or as marker for liquids, especially oils.

A further aspect of the present invention relates to a color converter comprising
(i) a compound of formula (I) as defined above;
(ii) a polymeric matrix material selected from a polystyrene, polycarbonate, polyacrylate, polymethylmethacrylate, polymethacrylate, polyvinylpyrrolidone, polyvinyl acetate, polyvinyl chloride, polybutene, silicone, epoxy resin, polyvinyl alcohol, poly(ethylene vinylalcohol)-copolymer, polyacrylonitrile, polyvinylidene chloride, polystyrene acrylonitrile, polybutylene terephthalate, polyethylene terephthalate, a 2,5-furandicarboxylate polyester, polyvinyl butyrate, polyvinyl chloride, polyamides, polyoxymethylenes, polyimides, polyetherimides or mixtures thereof; and
(iii) optionally a light scattering agent.

A further aspect of the present invention relates to a near infrared light source, comprising
(i) a light source, selected from a blue LED, red LED or white LED; and
(ii) a color converter as defined above.

The compound of formula (I) as described herein provides several benefits, in particular high solubility and stability in the application medium. Moreover, certain compounds of formula (I) are outstandingly suitable as fluorescent dye so that they can be used as NIR compound emitting light comprising a wavelength of 650 to 1200 nm, especially 680 to 1200 nm due to their good solubility in the application medium and the high fluorescence quantum yield. In addition, the compound of formula (I) is outstandingly suitable as marker for liquids, especially oils, such as mineral oils due to its favorable application properties such as good solubility in the liquids, high molar extinction coefficient, good storage stability and good detectability even in very small amounts in the correspondingly marked liquids.

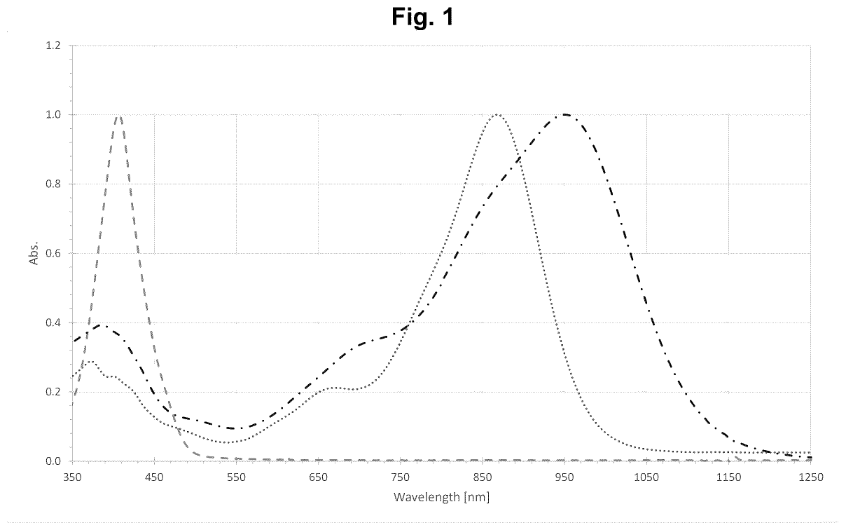

FIG. 1: Normalized UVNIS/NIR absorption spectra of dichlorosulfofluorescein

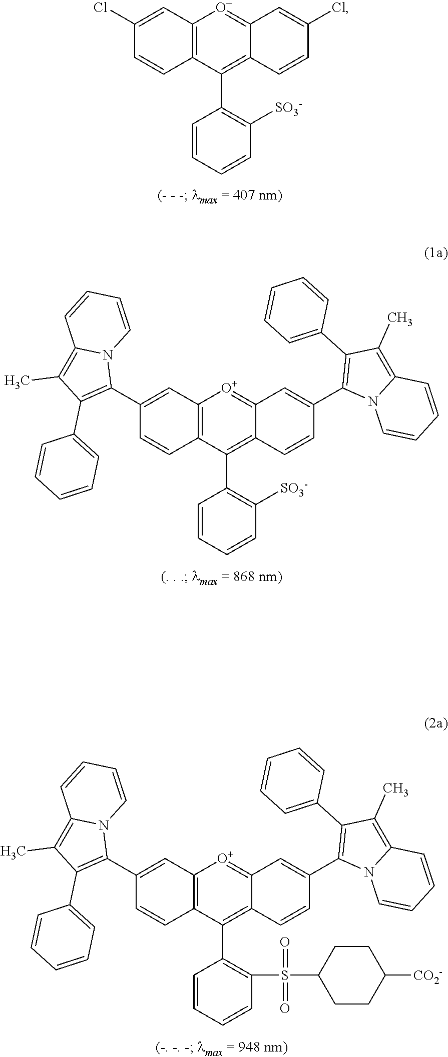

in dichloromethane solutions.

### DETAILED DESCRIPTION OF THE INVENTION

Here and throughout the specification, the term “near-infrared light” denotes light that ranges from 680 to 1700 nm, especially 680 to 1200 nm. “NIR I” as used herein refers to the region of the electromagnetic spectrum having wavelengths from about 680 to about 900 nm, while “NIR II” refers to that region having wavelengths from about 900 nm to about 1200 nm.

Here and throughout the specification, the term “visible light” denotes light that ranges from approximately 380 nm to 740 nm.

“Photoluminescent” as used herein refers to a molecule capable of absorbing a photon, in turn exciting an electron in the molecule to a higher electronic excited state, and then radiating a photon as light as the electron returns to a lower energy state. In one aspect, the NIR compounds and compositions disclosed herein are photoluminescent in the NIR region.

As used herein, “fluorescence quantum yield” (φ) refers to the ratio of photons absorbed to photons emitted through fluorescence.

“Stokes shift” as used herein refers to the difference between the maximum position in an absorption band for a compound and the maximum position of fluorescence emission for the same compound.

In one aspect, the NIR compounds disclosed herein have a Stokes shift of 150 nm or greater in the NIR region.

“Molar absorptivity,” “molar absorption coefficient,” “extinction coefficient,” and “molar attenuation coefficient” (s) refer to how strongly a chemical compound absorbs light at a given wavelength. Molar absorptivity is an intrinsic property of the compound; however, this coefficient varies with wavelength and solvent. Molar absorptivity is typically expressed in terms of absorption at a particular wavelength, such as the maximum position in the absorption band. Units are typically given as L/mol cm or M⁻¹ cm⁻¹. In one aspect, the disclosed NIR compounds have a high s in the NIR spectral region.

An “anionic counterion” is anion, or an anionic group associated with the cationic charge of the compound of formula (I); more particularly, the anionic counterion is chosen from i) halides, such as chloride or bromide; ii) nitrates; iii) sulfonates, including C₁-C₆alkylsulfonates: Alk-S(O)₂O⁻, such as methylsulfonate or mesylate and ethylsulfonate; iv) arylsulfonates: ArS(O)₂O⁻, such as benzenesulfonate and toluenesulfonate or tosylate; v) citrate; vi) succinate; vii) tartrate; viii) lactate; ix) alkylsulphates: Alk-O—S(O)O⁻ such as methysulphate and ethylsulphate; x) arylsulphates: Ar—O—S(O)O⁻ such as benzenesulphate and toluenesulphate; xi) alkoxysulphates: Alk-O—S(O)₂O⁻ such as methoxy sulphate and ethoxy sulphate; xii) aryloxy sulphates: Ar—OS(O)₂O⁻; (xiv) phosphate; xiii) phosphate; xiv) acetate; xv) triflate; and xvi) borates, such as tetrafluoroborate.

Here and throughout the specification, the term “halogen” denotes fluorine, bromine, chlorine or iodine, particularly chlorine, bromine or iodine.

The term “C₁-Cₙ-alkyl” denotes a group of linear or branched saturated hydrocarbon radicals having from 1 to n carbon atoms. For example, the term C₁-C₂₄-alkyl denominates a group of linear or branched saturated hydrocarbon radicals having from 1 to 24 carbon atoms, while the term C₁-C₄-alkyl denominates a group of linear or branched saturated hydrocarbon radicals having from 1 to 4 carbon atoms, the term C₅-C₂₀ alkyl denominates a group of linear or branched saturated hydrocarbon radicals having from 5 to 20 carbon atoms and the term C₆-C₂₀-alkyl denominates a group of linear or branched saturated hydrocarbon radicals having from 6 to 20 carbon atoms. Examples of alkyl include but are not limited to methyl, ethyl, n-propyl, isopropyl, n-butyl, sec-butyl, isobutyl, tert-butyl, 2-methylpropyl (isopropyl), 1,1-dimethylethyl (tert-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 1,1,3,3-tetramethylbutyl (tert-octyl), nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl and in case of nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl their isomers, in particular mixtures of isomers such as “isononyl”, “isodecyl”. Examples of C₁-C₄-alkyl are for example methyl, ethyl, propyl, 1-methylethyl, butyl, 1-methylpropyl, 2-methylpropyl or 1,1-dimethylethyl.

The term “C₁-C₂₄fluoroalkyl” as used herein denotes straight-chain or branched C₁-C₂₄ alkyl as defined above, where some or all of the hydrogen atoms in these groups may be replaced by fluorine above. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl and pentafluoroethyl.

The term “C₁-C₂₄alkoxy” as used herein denotes straight-chain or branched C₁-C₂₄ alkyl as defined above bound to the remainder of the molecule through an oxygen. Examples for C₁-C₄-alkoxy are methoxy, ethoxy, n-propoxy, 1-methylethoxy, butoxy, 1-methylpropoxy, 2-methylpropoxy and 1,1-dimethylethoxy.

The term “C₃-C₁₀cycloalkyl” as used herein denotes a mono-, bi- or tricyclic cycloalkyl radical which is unsubstituted or substituted by one or more radicals R⁷, for example 1, 2, 3 or 4 R⁷ radicals. Examples of C₃-C₁₀cycloalkyl include but are not limited to cyclopropyl, cyclobutyl, cyclopentyl, cyclohexyl, methylcyclohexyl, dimethylcyclohexyl, cycloheptyl, cyclooctyl, and norbornyl (=bicyclo[2.2.1]heptyl).

The term “C₆-C₁₀-aryl” as used herein denotes phenyl or naphthyl.

The term “heteroaryl”, especially C₂-C₁₄heteroaryl, as used herein refers to heteroaromatic, monocyclic, bicyclic or tricyclic condensed system with 5, 6, 7, 8, 9, 10, 11, 12, 13 or 14 ring members in which at least one of the rings is aromatic and which contains 1, 2, 3 or 4 heteroatoms selected from N, S or O. Monocyclic hetaryl groups are preferably 5- or 6-membered hetaryl groups comprising 1, 2 or 3 heteroatoms selected from O, S or N such as 2-furyl (furan-2-yl), 3-furyl (furan-3-yl), 2-thienyl (thiophen-2-yl), 3-thienyl (thiophen-3-yl), 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Bicyclic throughout aromatic heteroaryl is 9- or 10-membered and contains 1, 2, 3 or 4 heteroatoms selected from O, S or N. Examples are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl, benzothiadiazolyl, benzoxazinyl, benzopyrazolyl, benzimidazolyl, benzotriazolyl, benzotriazinyl.

The term “C₆-C₁₀aryl-C₁-C₁₀alkylene” (which may also be referred to as aralkyl) as used herein refers to C₆-C₁₀aryl-substituted alkyl radicals having at least one unsubstituted or substituted aryl group, as defined herein. The alkyl group of the aralkyl radical may be interrupted by one or more nonadjacent groups selected from O, S and —NR¹², wherein R^{12′} is as defined below, or above. C₆-C₁₀-aryl-C₁-C₁₀-alkylene is preferably phenyl-C₁-C₁₀-alkylene, more preferably phenyl-C₁-C₄-alkylene, for example benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl or 4-phenbut-2-yl;
preferably benzyl and 2-phenethyl.

In a preferred embodiment the compound of formula (I) is a compound of formula

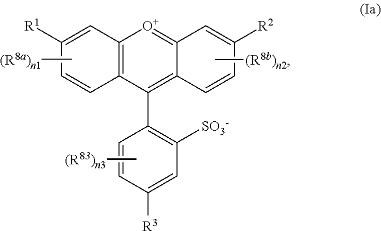

wherein n1, n2, n3, R¹, R², R³, R^{8a}, R^{8b} and R^{8c} are defined above, or below.

In said embodiment n1, n2 and n3 are preferably 0.

Accordingly, compounds of formula

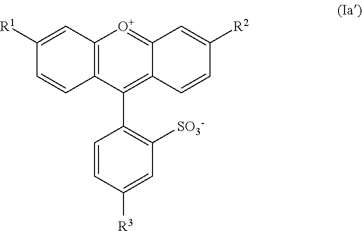

are more preferred.

R³ is preferably hydrogen.

R¹ and R² are preferably a group of formula

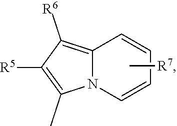

especially

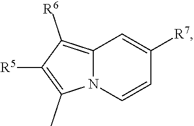

wherein R⁵, R⁶ and R⁷ are defined above, or below.

R⁷ is preferably a group —NR¹⁰R¹¹, wherein R¹⁰ and R¹¹ independently of each other a C₁-C₂₄alkyl group, a hydroxy-C₁-C₂₄alkyl group, a C₆-C₁₀-aryl group, which may be substituted with one or more substituents R¹⁴, or a C₃-C₁₀-cycloalkyl group, substituted with one or more substituents R¹⁴; or R¹⁰ and R¹¹ together with the nitrogen to which they are bonded form a five, or six membered ring system, which may be interrupted by —O—, —S—, or —NR¹²′— and may be part of a fused ring system, which may be substituted with one or more substituents R¹⁴.

Examples of groups —NR¹⁰R¹¹ are a phenylmethylamino group, a diphenylamino group, a dimethylamino group, a methylethylamino group, a 2-hydroxyethylmethylamino group, a 2-hydroxyethylethylamino group, a di-2-hydroxyethylamino group, a 2-ethylhexylmethylamino group, a 2-ethylhexylethylamino group, a 3-propylhepthylmethylamino group and a 3-propylhepthylethylamino group.

Examples of groups —NR¹⁰R¹¹, wherein R¹⁰ and R¹¹ together with the nitrogen to which they are bonded form a five, or six membered ring system are shown below:

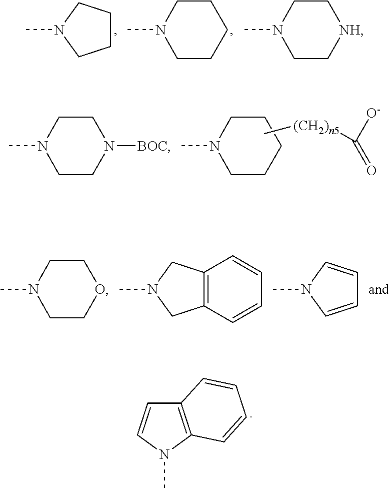

BOC is tert-butyloxycarbonyl. n5 is preferably 0, or an integer 1 to 5, more preferably 0, or 1.

R¹ and R² are more preferably a group of formula

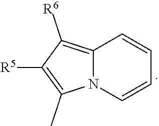

R⁵ is preferably a C₆-C₁₀aryl group, which may be substituted with one or more substituents R¹⁴; such as, for example, a phenyl group, a 4-cyano phenyl group, a 4-trifluoromethylphenyl group, a 4-nitrophenyl group, a 3,5-bis-trifluoromethylphenyl group, a 4-hydroxyphenyl group, or a 4-methoxyphenyl group, especially a phenyl group.

R⁶ is preferably a C₁-C₄alkyl group, or a CF₃ group, especially a methyl group.

R¹ and R² are even more preferably a group of formula

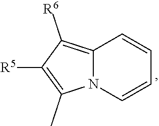

wherein R⁵ is a phenyl group, a 4-cyano phenyl group, a 4-trifluoromethylphenyl group, a 4-nitrophenyl group, a 3,5-bis-trifluoromethylphenyl group, a 4-hydroxyphenyl group, or a 4-methoxyphenyl group, especially a phenyl group; and
R⁶ is a C₁-C₄alkyl group, or a CF₃ group.

Most preferred R⁵ is a phenyl group and R⁶ is a methyl group, i.e. R¹ and R² represent a group of formula

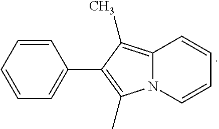 [SMILES: Cc1c(-c2ccccc2)c(C)n2ccccc12]

Examples of compounds of formula (Ia′), wherein R³ is hydrogen, are shown in the table below:

| Cpd. | R¹ = R² |
|---|---|
| 1a | 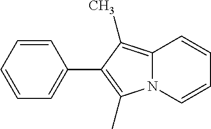 [SMILES: Cc1c(-c2ccccc2)c(C)n2ccccc12] |
| 1b | 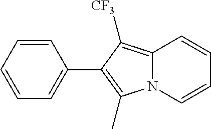 [SMILES: Cc1c(-c2ccccc2)c(C(F)(F)F)c2ccccn12] |

In another preferred embodiment the compound of formula (I) is a compound of formula

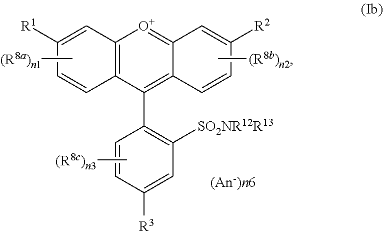

wherein n6 is 0, or 1, An⁻ is an anion and n1, n2, n3, R¹, R², R³, R^{8a}, R^{8b}, R^{8c}, R¹² and R¹³ are defined above, or below.

In said embodiment n1, n2 and n3 are preferably 0.

Accordingly, compounds of formula

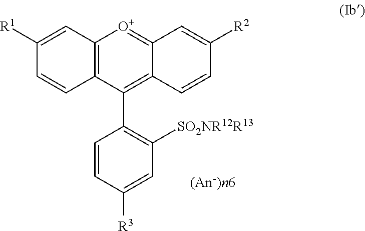

are more preferred. n6 is 0, or 1, An⁻ is an anion and R¹, R², R³, R¹² and R¹³ are defined above, or below.

If, for example, —NR¹²R¹³ represent a group of formula

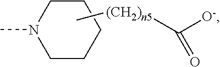

n6 is 0.

If the group —NR¹²R¹³ is not negatively charged, n6 is 1. The anion is chosen from i) halides, such as chloride or bromide; ii) nitrates; iii) sulfonates, including C₁-C₆alkylsulfonates: Alk-S(O)₂O⁻, such as methylsulfonate or mesylate and ethylsulfonate; iv) arylsulfonates: ArS(O)₂O⁻, such as benzenesulfonate and toluenesulfonate or tosylate; v) citrate; vi) succinate; vii) tartrate; viii) lactate; ix) alkylsulphates: Alk-O—S(O)O⁻ such as methysulphate and ethylsulphate; x) arylsulphates: Ar—O—S(O)O⁻ such as benzenesulphate and toluenesulphate; xi) alkoxysulphates: Alk-O—S(O)₂O⁻ such as methoxy sulphate and ethoxy sulphate; xii) aryloxy sulphates: Ar—OS(O)₂O⁻; (xiv) phosphate; xiii) phosphate; xiv) acetate; xv) triflate; and xvi) borates, such as tetrafluoroborate.

R³ is preferably hydrogen.

R¹ and R² are preferably a group of formula

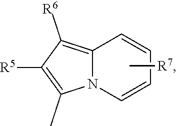

especially

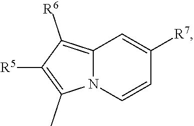

wherein R⁵, R⁶ and R⁷ are defined above, or below.

R⁷ is preferably a group —NR¹⁰R¹¹, wherein R¹⁰ and R¹¹ independently of each other a C₁-C₂₄alkyl group, a hydroxy-C₁-C₂₄alkyl group, a C₆-C₁₀-aryl group, which may be substituted with one or more substituents R¹⁴, or a C₃-C₁₀-cycloalkyl group, substituted with one or more substituents R¹⁴; or R¹⁰ and R¹¹ together with the nitrogen to which they are bonded form a five, or six membered ring system, which may be interrupted by —O—, —S—, or —NR¹²′— and may be part of a fused ring system, which may be substituted with one or more substituents R¹⁴.

Examples of groups —NR¹²R¹³ are a phenylmethylamino group, a diphenylamino group, a dimethylamino group, a methylethylamino group, a 2-hydroxyethylmethylamino group, a 2-hydroxyethylethylamino group, a di-2-hydroxyethylamino group, a 2-ethylhexylmethylamino group, a 2-ethylhexylethylamino group, a 3-propylhepthylmethylamino group and a 3-propylhepthylethylamino group.

Examples of groups —NR¹²R¹³, wherein R¹² and R¹³ together with the nitrogen to which they are bonded form a five, or six membered ring system are shown below:

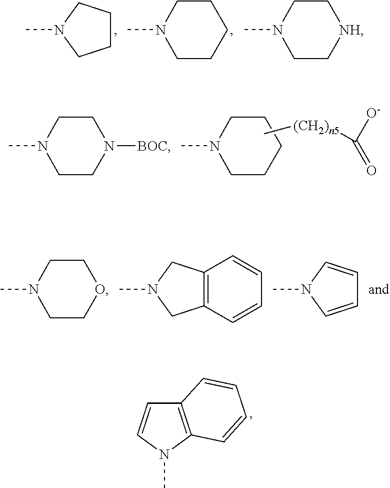

especially

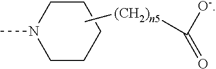

R¹ and R² are more preferably a group of formula

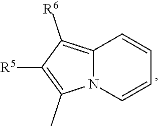

R⁵ is preferably a C₆-C₁₀-aryl group, which may be substituted with one or more substituents R¹⁴; such as, for example, a phenyl group, a 4-cyano phenyl group, a 4-trifluoromethylphenyl group, a 4-nitrophenyl group, a 3,5-bis-trifluoromethylphenyl group, a 4-hydroxyphenyl group, or a 4-methoxyphenyl group, especially a phenyl group.

R⁶ is preferably a C₁-C₄alkyl group, or a CF₃ group, especially a methyl group.

R¹ and R² are even more preferably a group of formula

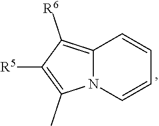

wherein R⁵ is a phenyl group, a 4-cyano phenyl group, a 4-trifluoromethylphenyl group, a 4-nitrophenyl group, a 3,5-bis-trifluoromethylphenyl group, a 4-hydroxyphenyl group, or a 4-methoxyphenyl group, especially a phenyl group; and
R⁶ is a C₁-C₄alkyl group, or a CF₃ group.

Most preferred R⁵ is a phenyl group and R⁶ is a methyl group, i.e. R¹ and R² represent a group of formula

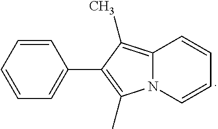 [SMILES: Cc1c(-c2ccccc2)c(C)n2ccccc12]

Examples of compounds of formula (Ib′) are compounds of formula

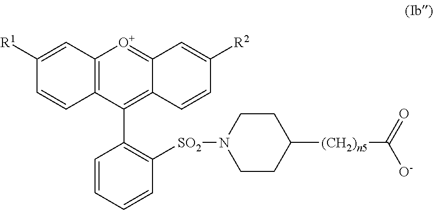

shown in the table below.

| Cpd. | R¹ = R² | n5 |
|---|---|---|
| 2a | 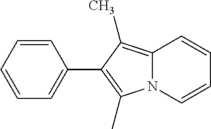 [SMILES: Cc1c(-c2ccccc2)c(C)n2ccccc12] | 0 |
| 2a | ″ | 1 |
| 2c | 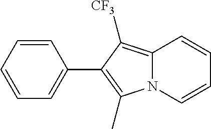 [SMILES: Cc1c(-c2ccccc2)c(C(F)(F)F)c2ccccn12] | 0 |
| 2d | ″ | 1 |

A process for the production of a compound of formula

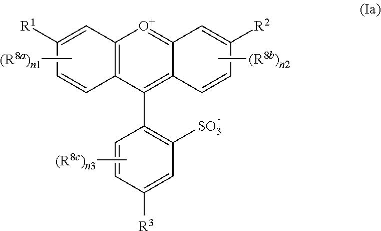

comprises
i) reacting a compound of formula

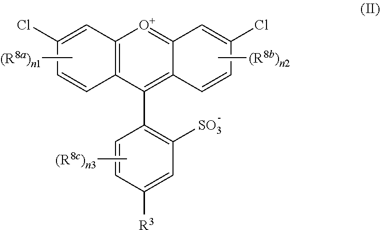

with a compound of formula

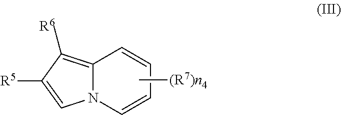

in the presence of diisopropylethylamine in a solvent, especially n-butanol, at a temperature of 20° C. to the boiling point of the solvent, wherein n1, n2, n3, n4, R¹, R², R³, R⁵, R⁶, R⁷, R^{8a}, R^{8b} and R^{8c} are defined above.

The compound of formula R

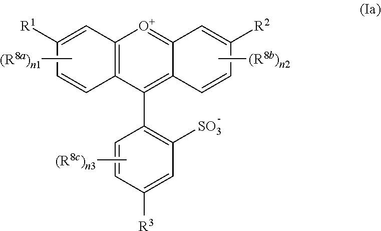

may be converted in a compound of formula

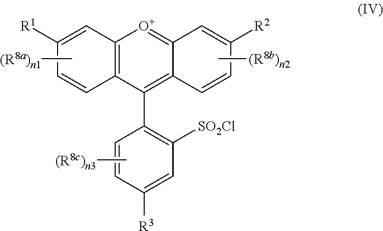

by reaction with phosphoryl chloride (O═P(Cl)₃).

Compounds of formula

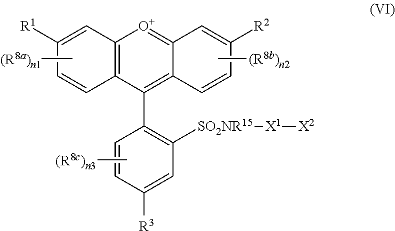

may be obtained by reacting the compound of formula (IV) with a compound of formula HNR¹⁵—X¹—X² (V), wherein X² is a reactive functional group.

Compounds of formula

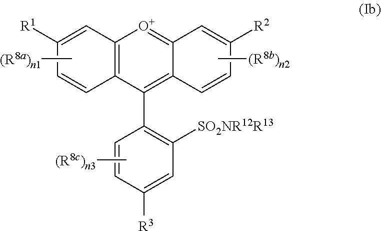

may be obtained by reacting the compound of formula (IV) with a compound of formula HNR¹²R¹³ (VII).

The compound of formula (VI), wherein X² is a reactive functional group, may be converted into a compound of formula (VI), wherein X² is a conjugated substance.
X¹ is a linking group;
X² is a reactive functional group,
R¹⁵ is hydrogen, or a C₁-C₄alkyl group; and
n1, n2, n3, R¹, R², R³, R^{8a}, R^{8b}, R^{8c}, R¹² and R¹³ are defined above.

Preferred X¹ moieties have 1-20 nonhydrogen atoms selected from the group consisting of C, N, O, P, and S; and are composed of any combination of ether, thioether, amine, ester, carboxamide, sulfonamide, hydrazide bonds and aromatic or heteroaromatic bonds. X¹ preferably contains 4-10 nonhydrogen atoms, including one or two heteroatoms. Examples of X¹ include substituted or unsubstituted polymethylene, arylene, alkylenarylene, arylenealkylene, or arylenethio. In one embodiment, X¹ is or incorporates the formula —(CH₂)ₐ(CONH(CH₂)_{b})_{z}—, where a has any value from 0-5, b has any value from 1-5 and z is 0 or 1.

X² is an acrylamide, an activated ester of a carboxylic acid, an acyl azide, an acyl nitrile, an aldehyde, an alkyl halide, an amine, an anhydride, an aniline, an aryl halide, an azide, an aziridine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is meant chemically reactive platinum complexes as described in U.S. Pat. No. 5,714,327.

Preferably, X² is a phosphoramidite, a succinimidyl ester of a carboxylic acid, a haloacetamide, a hydrazine, an isothiocyanate, a maleimide group, a perfluorobenzamido, or an azidoperfluorobenzamido group. More preferably, X² is a phosphoramidite, a reactive platinum complex, or a succinimidyl ester of a carboxylic acid. Where X² is a reactive platinum complex, it is typically a haloplatinate.

If the reactive group is a photoactivatable group, such as an azide, diazirinyl, azidoaryl, azidoperfluoroaryl, or a psoralen derivative, the compound of the invention typically becomes chemically reactive only after illumination with light of an appropriate wavelength.

If X² is an activated ester of a carboxylic acid (such as a succinimidyl ester), the reactive compound of the invention is particularly useful for preparing conjugates of proteins, polysaccharides, lipids, nucleotides, or amino-modified oligonucleotides or haptens. If X² is a maleimide or haloacetamide, the reactive compound of the invention is particularly useful for conjugation to thiol-containing substances. If X² is a hydrazide, the reactive compound of the invention is particularly useful for conjugation to periodate-oxidized carbohydrates and glycoproteins. If X² is a phosphoramidite, the reactive compound of the invention is particularly useful for the preparation of conjugates of oligonucleotides.

The conjugated substance (S_{C}) is a natural or synthetic amino acid (including those that are protected or are substituted by phosphates, carbohydrates, or C₁ to C₂₂ carboxylic acids), or is a natural or synthetic polymer of amino acids such as a peptide or protein that is optionally derivatized by a chemical protecting group; a natural or synthetic nucleic acid base, nucleoside, nucleotide or a nucleic acid polymer, a carbohydrate that is typically a natural or synthetic polysaccharide, lipid (typically having 6-60 carbons), including glycolipids, phospholipids, sphingolipids, glycerides, and steroids. Where the lipid is a phospholipid.

Examples of groups —SO₂NR¹²′—X¹—X², wherein X² is a reactive functional group, are a group of formula

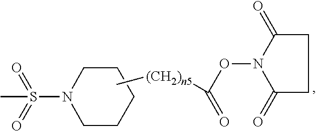

such as, for example,

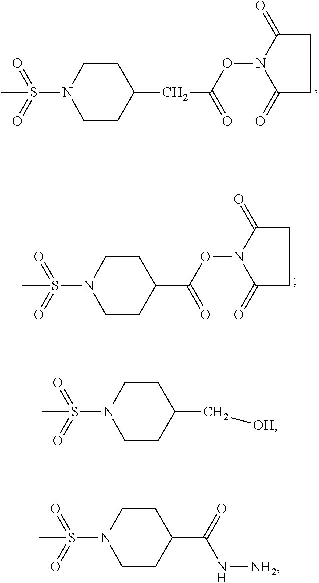 [SMILES: CS(=O)(=O)N1CCC(CC(=O)ON2C(=O)CCC2=O)CC1 ; CS(=O)(=O)N1CCC(C(=O)ON2C(=O)CCC2=O)CC1 ; CS(=O)(=O)N1CCC(CO)CC1 ; CS(=O)(=O)N1CCC(C(=O)NN)CC1]

or —SO₂—N(CH₃)(CH₂)₆OH.

The compounds of formula

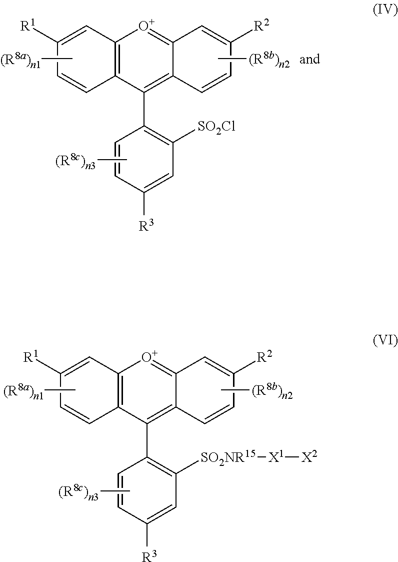

are new and form further subjects of the present invention. wherein
X¹ is a linking group;
X² is a reactive functional group, or a conjugated substance, and
R¹⁵ is hydrogen, or a C₁-C₄alkyl group; and
n1, n2, n3, n5, R¹, R², R³, R^{8a}, R^{8b} and R^{8c} are defined above. For n1, n2, n3, n5, R¹, R², R³, R^{8a}, R^{8b} and R^{8c} the same preferences apply as in case of the compounds of formula (I).

An example of a compound of formula (IV) is a compound of formula (IV), wherein R¹=R²=

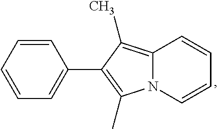 [SMILES: Cc1c(-c2ccccc2)c(C)n2ccccc12]

n1, n2 and n3 are 0 and R³ is hydrogen (cpd. (4)).

Examples of compounds of formula (VI), wherein X² is a reactive functional group, n1, n2 and n3 are 0, R³ is hydrogen, are shown in the table below:

| Cpd. | R¹ = R² | —SO₂NR^{12′}—X¹—X² |
|---|---|---|
| 3a | 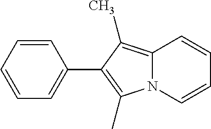 [SMILES: Cc1c(-c2ccccc2)c(C)n2ccccc12] | 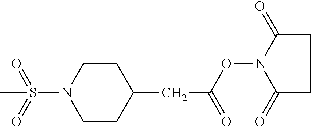 [SMILES: CS(=O)(=O)N1CCC(CC(=O)ON2C(=O)CCC2=O)CC1] |
| 3b | ″ | 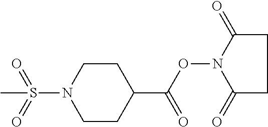 [SMILES: CS(=O)(=O)N1CCC(C(=O)ON2C(=O)CCC2=O)CC1] |
| 3c | ″ | 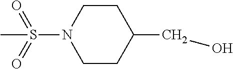 |
| 3d | ″ | 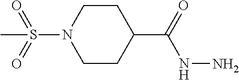 |
| 3e | ″ | —SO₂—N(CH₃)(CH₂)₆OH |

The compounds of the formula (I) according to the invention may be incorporated without any problem into organic and inorganic materials and are therefore suitable for a whole series of end uses, some of which will be listed by way of example below.

In general, the compounds of formula (I) are fluorescent dyes that absorb light having a wavelength in the range from 450 to 950 nm. They generally have their absorption maximum in the range from 600 to 880 nm. They generally emit light in a range from 680 to 1200 nm. The fluorescence light thus generated is advantageously detected with a semiconductor detector, especially with a silicon photodiode or a germanium photodiode. For these applications, it is important to use the compound of formula (I) in high concentration to convert as much of the absorbed light as possible.

The compounds of formula (I) are outstandingly suitable for homogeneously coloring high molecular weight organic and inorganic materials, in particular, for example, plastics, in particular thermoplastics, coatings and printing inks, and also oxidic layer systems.

NIR spectroscopy is a well-established technique for detecting both chemical and physical properties of various materials. For example, NIR spectroscopy may be used for a non-destructive food analysis or for a non-destructive plant analysis in agriculture. The compounds of formula (I) are also especially useful as fluorescent compound in a near infrared spectrometer apparatus for providing light having a wavelength in the range from 680 to 950 nm.

The compounds of formula (I) are also of interest as active components in photovoltaics. Thus, the present invention also relates to the use of the compounds of formula (I) in photovoltaic applications, especially in a fluorescent solar concentrator. The solar concentrator is based on solar cells and a polymeric matrix material comprising the compound of formula (I) and the solar cells are located at the outer edges of the polymeric material.

The compounds of formula (I) are also of interest as dye for laser applications.

The compounds of formula (I) are also of interest as semiconductor in organic electronic applications, especially as semiconductor in an organic field effect transistor or as semiconductor in an organic electroluminescent device. The compounds of formula (I) may also be used as semiconductor in dye-sensitized solar cells.

Moreover, the compounds of formula (I) are suitable as near infrared absorbers for heat management and as NIR laser beam-absorbent materials in the fusion treatment of plastics parts. These applications are described in detail, for example, in DE102004018547, WO02/77081 and WO04/05427.

Moreover, the compounds of formula (I) may also be used advantageously for laser marking and laser inscription. In this case, the laser light absorbed by the compound of formula (I) brings about heating of the plastic, which leads to it foaming or the conversion of a dye present in addition, and in this way gives rise to a marking or inscription.

The compounds of formula (I) are also of interest as labeling groups in detection methods, especially in diagnostic and analytical methods on biological samples, including living cells.

Accordingly, the present invention is directed to a composition comprising a compound of formula (I) and a pharmaceutically-acceptable carrier; and a method for imaging a biological sample, the method comprising:
(a) contacting the biological sample with the composition;
(b) exposing the biological sample and the composition to VIS/NIR radiation; and
(c) observing NIR fluorescence emission in the biological sample.

The biological sample comprises preferably an organelle, a cell, a tissue, an organ, or any combination thereof.

The compounds of formula (I) are also of interest for use in an ink for machine readability and/or security applications.

The compounds of formula (I) owing to their pronounced absorption in the near infrared region of the electromagnetic spectrum, are also of interest for obtaining markings and inscriptions which absorb near infrared light and are invisible to the human eye. Thus, the present invention also relates to the use of the compound of formula (I) as defined above for brand protection or as marker for liquids. Useful liquids which can be marked with the compounds of the formula (I) preferably include oils such as mineral oils, vegetable and animal fatty oils, and ethereal oils.

Examples of such oils are natural oils such as olive oil, soybean oil or sunflower oil, or natural or synthetic motor oils, hydraulic oils or transmission oils, for example motor vehicle oil or sewing machine oil, or brake fluids and mineral oils which, according to the invention, comprise gasoline, kerosene, diesel oil and also heating oil. Particular preference is given to mineral oils such as gasoline, kerosene, diesel oil or heating oil, in particular gasoline, diesel oil or heating oil. Particularly advantageously, the above-mentioned compounds of the formula (I) are used as markers for mineral oils in which labeling is simultaneously required, for example for tax reasons. In order to minimize the costs of labeling, but also in order to minimize possible interactions of the marked mineral oils with any other ingredients present, such as polyisobuteneamine (PIBA), efforts are made to minimize the amount of markers. A further reason to minimize the amount of markers may be to prevent their possible harmful influences, for example on the fuel intake and exhaust gas outlet region of internal combustion engines.

The compounds of the formula (I) to be used as markers are added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb.

The compounds of the formula (I) may if appropriate also be used in a mixture with other markers/dyes.

To mark the liquids, the compounds are generally added in the form of solutions. Especially in the case of mineral oils, suitable solvents for providing these stock solutions are preferably aromatic hydrocarbons such as toluene, xylene or relatively high-boiling aromatics mixtures.

The compounds of formula (I) can also be used in the form of a mixture, comprising the compound of formula (I) and at least one further IR absorber different from the compound of formula (I). Suitable further IR absorbers are in principle all known classes of IR absorbers that are compatible with the compound of formula (I). Preferred further IR absorbers are selected from polymethines, phthalocyanines, naphthalocyanines, quinone-diimmonium salts, aminium salts, rylenes, inorganic IR absorbers and mixtures thereof. Further polymethine IR absorbers are preferably selected from cyanines, squaraines, croconaines and mixtures thereof. Further inorganic IR absorbers are preferably selected from indium tin oxide, antimony tin oxide, lanthanum hexaboride, tungsten bronzes, copper salts etc.

The IR absorbers can be generally used in a concentration of from 10 ppm to 25%, preferably 100 ppm to 10%, depending on the chosen application.

The compounds of formula (I) and IR absorber mixtures are especially suitable for security printing.

Security printing is the field that deals with the printing of items such as currency, passports, tamper-evident labels, stock certificates, postage stamps, identity cards, etc. The main goal of security printing is to prevent forgery, tampering or counterfeiting.

In the field of automated banknote processing, IR-absorption plays an important role. Most of the actually circulating currency carries not only visibly coloured printings, but also specific features which are only detectable in the infrared part of the spectrum. Generally, these IR-features are implemented for use by automatic currency processing equipment, in banking and vending applications (automatic teller machines, automatic vending machines, etc.), in order to recognize a determined currency bill and to verify its authenticity, in particular to discriminate it from replicas made by colour copiers.

Accordingly, the present invention also relates to a method of detecting the authenticity of a security document as defined above, or below, comprising the steps of:
a) measuring an absorbance, reflectance or transmittance spectrum of the security document in the VIS/NIR range of the electromagnetic spectrum; and
b) comparing the spectrum measured under a) and/or information derived therefrom with a corresponding spectrum and/or information of an authentic security element.

All security documents are required to have good stability and durability. In the case of bank notes, these requirements are extreme, as bank notes are subjected to toughest use conditions by the public—they are subjected to material stress by folding, crumpling etc., subjected to abrasion, exposed to weather, exposed to bodily fluids such as perspiration, laundered, dry-cleaned, ironed etc.—and, after having been subjected to this, are expected to be as legible as when they started. Furthermore, it is essential that the documents nevertheless should have a reasonable life time, ideally of some years, despite suffering the afore-mentioned conditions. During this time, the documents, and thus the inks on them (including invisible security markings), should be resistant to fading or colour change. Hence, any ink used in a security printing process should, when cured, be robust, water-resistant, resistant to various chemicals and flexible. Moreover, as certain states are moving away from the use of paper as the substrate for bank notes, the employed printing ink formulations should be useable on plastics as well as paper. The compounds of formula (I) because of its unique application properties are especially suitable for printing ink formulations that are employed for security printing and in particular for bank notes, identity cards, passports, tax stamps, stock certificates, credit cards, labels etc.

In security printing, the IR absorber is added to a printing ink formulation. Suitable printing inks are water-based, oil-based or solvent-based printing inks, based on pigment or dye, for inkjet printing, gravure printing, flexographic printing, screen printing, intaglio printing, offset printing, laser printing or letterpress printing and for use in electrophotography. Printing inks for these printing processes usually comprise solvents, binders, and also various additives, such as plasticizers, antistatic agents or waxes. Printing inks for offset printing, intaglio printing and letterpress printing are usually formulated as high-viscosity paste printing inks, whereas printing inks for inkjet printing, flexographic printing and gravure printing are usually formulated as liquid printing inks with comparatively low viscosity.

In the context of the present invention, the expression “printing ink” also encompasses formulations that in addition to at least one IR absorber of the general formula (I) comprise a colorant. The expression “printing ink” also encompasses printing lacquers that comprise no colorant.

The printing ink formulation for security printing according to the invention preferably comprises
a) a compound of formula (I) as defined above,
b) a polymeric binder,
c) a solvent,
d) optionally at least one colorant, and
e) optionally at least one further additive.

Suitable components of printing inks are conventional and are well known to those skilled in the art. Examples of such components are described in “Printing Ink Manual”, fourth edition, Leach R. H. et al. (eds.), Van Nostrand Reinhold, Wokingham, (1988). Details of printing inks and their formulation are also disclosed in “Printing Inks”-Ullmann's Encyclopedia of Industrial Chemistry, Sixth Edition, 1999 Electronic Release. A formulation of an IR-absorbing intaglio ink formulation is described in US 20080241492 A1. The disclosure of the afore-mentioned documents is incorporated herein by reference.

The printing ink formulation according to the invention contains in general from 0.0001 to 25% by weight, preferably from 0.001 to 15% by weight, in particular from 0.01 to 5% by weight, based on the total weight of the printing ink formulation, of component a).

The compound of formula (I) is present in the printing ink formulation in dissolved form or in solid form (in a finely divided state).

The printing ink formulation according to the invention contains in general from 5 to 74% by weight, preferably from 10 to 60% by weight, more preferably from 15 to 40% by weight, based on the total weight of the printing ink formulation, of component b).

Suitable polymeric binders b) for the printing ink formulation according to the invention are for example selected from natural resins, phenol resin, phenol-modified resins, alkyd resins, polystyrene homo- and copolymers, terpene resins, silicone resins, polyurethane resins, urea-formaldehyde resins, melamine resins, polyamide resins, polyacrylates, polymethacrylates, chlorinated rubber, vinyl ester resins, acrylic resins, epoxy resins, nitrocellulose, hydrocarbon resins, cellulose acetate, and mixtures thereof.

The printing ink formulation according to the invention can also comprise components that form a polymeric binder by a curing process. Thus, the printing ink formulation according to the invention can also be formulated to be energy-curable, e.g. able to be cured by UV light or EB (electron beam) radiation. In this embodiment, the binder comprises one or more curable monomers and/oligomers. Corresponding formulations are known in the art and can be found in standard textbooks such as the series “Chemistry & Technology of UV & EB Formulation for Coatings, Inks & Paints”, published in 7 volumes in 1997-1998 by John Wiley & Sons in association with SITA Technology Limited.

Suitable monomers and oligomers (also referred to as prepolymers) include epoxy acrylates, acrylated oils, urethane acrylates, polyester acrylates, silicone acrylates, acrylated amines, and acrylic saturated resins. Further details and examples are given in “Chemistry & Technology of UV & EB Formulation for Coatings, Inks & Paints”, Volume II: Prepolymers & Reactive Diluents, edited by G Webster.

If a curable polymeric binder is employed, it may contain reactive diluents, i.e. monomers which act as a solvent and which upon curing are incorporated into the polymeric binder. Reactive monomers are typically chosen from acrylates or methacrylates, and can be monofunctional or multifunctional. Examples of multifunctional monomers include polyester acrylates or methacrylates, polyol acrylates or methacrylates, and polyether acrylates or methacrylates.

In the case of printing ink formulations to be cured by UV radiation, it is usually necessary to include at least one photoinitiator to initiate the curing reaction of the monomers upon exposure to UV radiation. Examples of useful photoinitiators can be found in standard textbooks such as “Chemistry & Technology of UV & EB Formulation for Coatings, Inks & Paints”, Volume III, “Photoinitiators for Free Radical Cationic and Anionic Polymerisation”, 2nd edition, by J. V. Crivello & K. Dietliker, edited by G. Bradley and published in 1998 by John Wiley & Sons in association with SITA Technology Limited. It may also be advantageous to include a sensitizer in conjunction with the photoinitiator in order to achieve efficient curing.

The printing ink formulation according to the invention contains in general from 1 to 94.9999% by weight, preferably from 5 to 90% by weight, in particular from 10 to 85% by weight, based on the total weight of the printing ink formulation, of a solvent c).

Suitable solvents are selected from water, organic solvents and mixtures thereof. For the purpose of the invention, reactive monomers which also act as solvents are regarded as part of the afore-mentioned binder component b).

Examples of solvents comprise water; alcohols, e.g. ethanol, 1-propanol, 2-propanol, ethylene glycol, propylene glycol, diethylene glycol and ethoxy propanol; esters, e.g. ethyl acetate, isopropyl acetate, n-propyl acetate and n-butyl acetate; hydrocarbons, e.g. toluene, xylene, mineral oils and vegetable oils, and mixtures thereof.

The printing ink formulation according to the invention may contain an additional colorant d). Preferably, the printing ink formulation contains from 0 to 25% by weight, more preferably from 0.1 to 20% by weight, in particular from 1 to 15% by weight, based on the total weight of the printing ink formulation, of a colorant d).

Suitable colorants d) are selected conventional dyes and in particular conventional pigments. The term “pigment” is used in the context of this invention comprehensively to identify all pigments and fillers, examples being colour pigments, white pigments, and inorganic fillers. These include inorganic white pigments, such as titanium dioxide, preferably in the rutile form, barium sulfate, zinc oxide, zinc sulfide, basic lead carbonate, lithopones (zinc sulfide+barium sulfate), or coloured pigments, examples being iron oxides, bismuth vanadates, lead chromates, lead molybdates, iron blue, Cobalt blue, Cobalt green, Ni-rutile yellow, Cr-rutil yellow, zinc yellow, zinc green, ultramarine, manganese black, antimony black, manganese violet, carbon black, graphite. Besides the inorganic pigments the printing ink formulation of the invention may also comprise organic colour pigments, examples being Monoazo, Disazo, β-Naphthol, Naphthol AS, Azo pigment Lakes, Benzimidazolone, Metal complex pigments, Isoindolinone, Isoindoline, Phthalocyanine, Quinacridone, Perylene, perinone, Diketopyrrolo-Pyrrol, Thioindigo, Anthraquinone, Anthrapyrimidine, Indanthrone, Flavanthrone, Pyranthrone, Dioxazine, Triarylcarbonium, Quinophthalone. Also suitable are synthetic white pigments with air inclusions to increase the light scattering, such as the Rhopaque® dispersions. Suitable fillers are, for example, aluminosilicates, such as feldspars, silicates, such as kaolin, talc, mica, magnesite, alkaline earth metal carbonates, such as calcium carbonate, in the form for example of calcite or chalk, magnesium carbonate, dolomite, alkaline earth metal sulfates, such as calcium sulfate, silicon dioxide, etc.

The printing ink formulation according to the invention may contain at least one additive e). Preferably, the printing ink formulation contains from 0 to 25% by weight, more preferably from 0.1 to 20% by weight, in particular from 1 to 15% by weight, based on the total weight of the printing ink formulation, of at least one component e).

Suitable additives (component e)) are selected from plasticizers, waxes, siccatives, antistatic agents, chelators, antioxidants, stabilizers, adhesion promoters, surfactants, flow control agents, defoamers, biocides, thickeners, etc. and combinations thereof. These additives serve in particular for fine adjustment of the application-related properties of the printing ink, examples being adhesion, abrasion resistance, drying rate, or slip.

In particular, the printing ink formulation for security printing according to the invention preferably contains
a) 0.0001 to 25% by weight of the compound of formula (I),
b) 5 to 74% by weight of at least one polymeric binder,
c) 1 to 94.9999% by weight of at least one a solvent,
d) 0 to 25% by weight of at least one colorant, and
e) 0 to 25% by weight of at least one further additive,

wherein the sum of components a) to e) adds up to 100%.

The printing ink formulations according to the invention are advantageously prepared in a conventional manner, for example by mixing the individual components. As mentioned earlier, the compound of formula (I) is present in the printing ink formulations in a dissolved or finely divided solid form. Additional colorants may be employed in the printing ink formulation of the invention or in a separate ink formulation. When additional colorants are to be employed in a separate formulation, the time of application of the printing ink formulation according to the invention is usually immaterial. The printing ink formulation according to the invention can for example be applied first and then be overprinted with conventional printing inks. But it is also possible to reverse this sequence or, alternatively, to apply the printing ink formulation according to the invention in a mixture with conventional printing inks. In every case the prints are readable with suitable light sources.

Primers can be applied prior to the printing ink formulation according to the invention. By way of example, the primers are applied in order to improve adhesion to the substrate. It is also possible to apply additional printing lacquers, e.g. in the form of a covering to protect the printed image. Additional printing lacquers may also be applied to serve aesthetic purposes, or serve to control application-related properties. By way of example, suitably formulated additional printing lacquers can be used to influence the roughness of the surface of the substrate, the electrical properties, or the water-vapour-condensation properties. Printing lacquers are usually applied in-line by means of a lacquering system on the printing machine employed for printing the printing ink formulation according to the invention.

The printing ink formulations according to the invention are also suitable for use in multilayer materials. Multilayer materials are e.g. composed of two or more plastics foils, such as polyolefin foils, metal foils, or metallised plastics foils, which are bonded to one another, by way of example, via lamination or with the aid of suitable laminating adhesives. These composites may also comprise other functional layers, such as odour-barrier layers or water-vapour barriers.

In addition, the present invention is directed to security documents, comprising a substrate and a compound of formula (I) as defined above, or security document, obtainable by a printing process, wherein the printing ink formulation as defined above is employed.

The compound of formula (I) and IR absorber mixtures are also especially suitable for laser welding of plastics.

The laser welding is preferably carried out using an Nd:YAG laser at 1064 nm or using a diode laser at 980 nm or 940 nm. The concentration of the new crystal form of compound (1) or an IR absorber mixtures is e.g. from 5 to 500 ppm, preferably from 10 to 200 ppm.

In laser welding, plastics components are welded to one another. The plastics components to be fused may have any shape. For example, at least one of the plastics components may be a film.

The compound of formula (I) is suitable for welding transparent at least translucent plastics materials. The employed plastics materials may be colourless or coloured. In principle, the plastics components to be fused may be composed of the same polymer or of different polymers. Preferably, the plastics components employed for laser welding are selected from thermoplastic polymers. However, it is also possible that neither of the plastics components to be fused is composed of thermoplastic; however, a coating of at least one part with a thermoplastic comprising the compound of formula (I) is required.

The plastics components employed for laser welding preferably comprise or consist of at least one polymer selected from polyolefins, polyolefin copolymers, polytetrafluoroethylenes, ethylene-tetrafluoroethylene copolymers, polyvinyl chlorides, polyvinylidene chlorides, polyvinyl alcohols, polyvinyl esters, polyvinyl alkanals, polyvinyl ketals, polyamides, polyimides, polycarbonates, polycarbonate blends, polyesters, polyester blends, poly(meth)acrylates, poly(meth)acrylate-styrene copolymer blends, poly(meth)acrylate-polyvinylidene difluoride blends, polyurethanes, polystyrenes, styrene copolymers, polyethers, polyether ketones and polysulfones and mixtures thereof.

Preference is given to matrix polymers from the group of the polyolefins, polyolefin copolymers, polyvinyl alkanals, polyamides, polycarbonates, polycarbonate-polyester blends, polycarbonate-styrene copolymer blends, polyesters, polyester blends, poly(meth)acrylates, poly(meth)acrylate-styrene copolymer blends, poly(meth)acrylate-polyvinylidene difluoride blends, styrene copolymers and polysulfones and mixtures thereof.

Particularly preferred polymers are transparent or at least translucent. Examples include: polypropylene, polyvinylbutyral, nylon-[6], nylon-[6,6], polycarbonate, polycarbonate-polyethylene terephthalate blends, polycarbonate-polybutylene terephthalate blends, polycarbonate-acrylonitrile/styrene/acrylonitrile copolymer blends, polycarbonate-acrylonitrile/butadiene/styrene copolymer blends, polymethyl methacrylate-acrylonitrile/butadiene/styrene copolymer blends (MABS), polyethylene terephthalate, polybutylene terephthalate, polymethyl methacrylate, impact-modified polymethyl methacrylate, polybutyl acrylate, polymethyl methacrylate-polyvinylidene difluoride blends, acrylonitrile/butadiene/styrene copolymers (ABS), styrene/acrylonitrile copolymers (SAN), polyphenylenesulfone and mixtures comprising 2 or more (e.g. 2, 3, 4, 5) of the afore-mentioned polymers.

Suitable polymer preparations for laser welding comprise
A) a thermoplastic matrix polymer suitable for forming the plastics parts,
B) the compound of formula (I) as defined before,
C) optionally at least one further additive.

Those polymer preparations for laser welding are likewise in accordance with the invention and are suitable for producing fusion-bonded plastics parts with the aid of laser radiation whose wavelength is outside the visible region.

Polymer preparations for laser welding may advantageously be produced by a conventional extrusion or kneading process. The components B), and, if present, C) may be mixed from the outset, in the weight ratio corresponding to the desired end concentration, with the matrix polymer A) (direct compounding), or a distinctly higher concentration of B) and, if present, C) may initially be selected and the concentrate formed (masterbatch) subsequently diluted with further matrix polymer A) in the course of the manufacture of the parts to be fused.

Suitable additives C) are UV stabilizers, antioxidants, processing plasticizers, etc.

In addition, the polymer preparations for laser welding may comprise at least one colorant for establishing a desired hue as additive, especially transparent organic pigments and in particular dyes, for example C.I. Pigment Yellow 109, 110, 128, 138, 139, 150, 151, 147, 180, 183, 185 192 and 196, C.I. Pigment Orange 70, C.I. Pigment Red 122, 149, 178 and 179, 181, 202, 263, C.I. Pigment Violet 19, 23, 37 and 29, C.I. Pigment Blue 15, 15:1, 15:3 and 15:4, 60, C.I. Pigment Green 7 and 36, C.I. Solvent Yellow 14, 21, 93, 130, 133, 145, 163, C.I. Solvent Red 52, 135, 195, 213, 214 and 225, C.I. Solvent Blue 35, 45, 67, 68, 97, 104, 122, 132, C.I. Solvent Violet 13, 46, 49, C.I. Solvent Green 3, 5 and 28, C.I. Solvent Orange 47, 60, 86, 114, and 163, C.I. Solvent Brown 35, 53, and also C.I. Disperse Yellow 54, 87, 201, C.I. Disperse Orange 30, C.I. Disperse Red 60 and C.I. Disperse Violet 57.

A further possible additive group is that of additives which likewise modify the visual appearance, the mechanical properties or else the tactile properties, for example matting agents, such as titanium dioxide, chalk, barium sulfate, zinc sulfide, fillers, such as nanoparticulate silicon dioxide, aluminium hydroxide, clay and other sheet silicates, glass fibers and glass spheres.

The present invention further provides a color converter comprising
(i) a compound of formula (I) as defined above;
(ii) a polymeric matrix material selected from a polystyrene, polycarbonate, polyacrylate, polymethylmethacrylate, polymethacrylate, polyvinylpyrrolidone, polyvinyl acetate, polyvinyl chloride, polybutene, silicone, epoxy resin, polyvinyl alcohol, poly(ethylene vinylalcohol)-copolymer, polyacrylonitrile, polyvinylidene chloride, polystyrene acrylonitrile, polybutylene terephthalate, polyethylene terephthalate, a 2,5-furandicarboxylate polyester, polyvinyl butyrate, polyvinyl chloride, polyamides, polyoxymethylenes, polyimides, polyetherimides or mixtures thereof; and
(iii) optionally a light scattering agent.

The concentration of the compound of formula (I) as defined above and, if appropriate, of further colorants in the polymer matrix is set as a function of the thickness of the color converter and the type of polymer. If a thin polymer layer is used, the concentration of the compound of formula (I) and, if appropriate the concentration of further colorants, is generally higher than in the case of a thick polymer layer. Preferably, the concentration of the compound of formula (I) according to the present invention is in the range of from 0.001 to 2% by weight, especially 0.001 to 1% by weight, based on the weight of the polymeric matrix material.

In one embodiment of the invention, the color converter does not comprise a light scattering agent.

In another embodiment of the invention, the color converter comprises a light scattering agent. In a preferred embodiment of the invention, the polymeric matrix material comprises scattering agents. Suitable light scattering agents are inorganic white pigments, for example titanium dioxide, barium sulfate, lithopone, zinc oxide, zinc sulfide, calcium carbonate with a mean particle size to DIN 13320 of 0.01 to 10 μm, preferably 0.1 to 1 μm, more preferably 0.15 to 0.4 μm. These light scattering agents are included typically in an amount of 0.01 to 2.0% by weight, preferably 0.05 to 1.0% by weight, more preferably 0.1 to 0.6% by weight, based in each case on the polymer of the layer comprising scattering bodies.

Examples of suitable organic light scattering agents include scattering polymers such as those based on poly(acrylates); poly (alkyl methacrylates), for example poly(methyl methacrylate) (PMMA); poly (tetrafluoroethylene) (PTFE); silicone-based scattering agents, for example hydrolyzed poly(alkyl trialkoxysilanes), and mixtures thereof. The size of these light scattering agents (average diameter-weight average) is usually in the range from 0.5 to 50 μm, preferably 1 to 10 μm. These light scattering agents are typically included in an amount of 1 to 10% by weight, based in each case on the polymer of the layer comprising scattering bodies. Useful light scattering agents are for example a mixture of 3 to 5% by weight of PMMA based scattering agent and 1.5 to 2% by weight of silicone based scattering agent.

Also suitable are light-scattering compositions which contain polymeric particles based on vinyl acrylate with a core/shell morphology in combination with TiO₂ as described in EP-A 634 445.

The polymeric matrix material can also comprise at least one further additive selected from an UV absorber, a hindered amine light stabilizer, flame retardant, UV stabilizer, thermal stabilizer, anti-oxidant, plasticizer, antifogging agent, nucleating agent, antistatic agent, filler or a reinforcing material, or combinations thereof.

Hindered amine light stabilizers, UV stabilizers and thermal stabilizers are known to those skilled in the art. Suitable antioxidants or free-radical scavengers are, for example, phenols, especially sterically hindered phenols, such as butylhydroxyanisole (BHA) or butylhydroxytoluene (BHT), or sterically hindered amines (HALS). Stabilizers of this kind are sold, for example, by BASF under the Irganox® trade name. In some cases, antioxidants and free-radical scavengers can be supplemented by secondary stabilizers, such as phosphites or phosphonites, as sold, for example, by BASF under the Irgafos® trade name.

Suitable UV absorbers are, for example, benzotriazoles, such as 2-(2-hydroxyphenyl)-2H-benzotriazole (BTZ), triazines, such as (2-hydroxyphenyl)-s-triazine (HPT), hydroxybenzophenones (BP) or oxalanilides. UV absorbers of this kind are sold, for example, by BASF under the Uvinul® trade name.

The color converter comprising the compound of formula (I) can be part of an agricultural foil, agricultural netting or a greenhouse screen or an illumination device. The color converter may be supported by glass. Likewise it is possible that the agricultural foil, agricultural netting or greenhouse screen consists of the color converter used according to the invention. The color converter according to the present invention can also be part of a near infrared light source.

Thus, a further object of the present invention relates to a near infrared light source, comprising
(i) a light source, selected from a blue LED, red LED or white LED; and
(ii) a color converter as defined above.

The near infrared light source may be part of a NIR-LED or a near infrared spectrometer.

The invention will be illustrated in detail by the examples.

### EXAMPLES

### Example 1

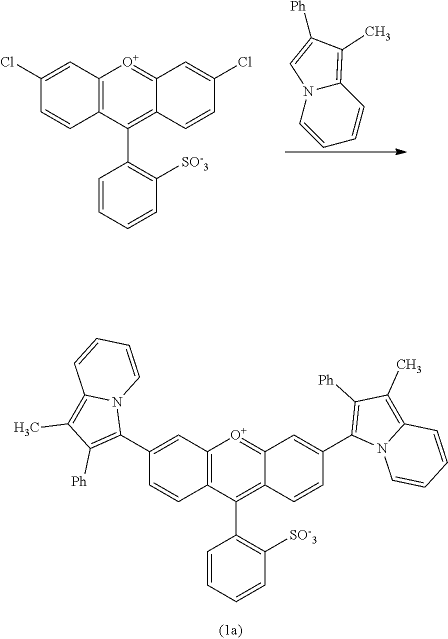

Under nitrogen atmosphere 10.0 parts of dichlorosulfofluorescin and 10.2 parts 1-methyl-2-phenylindolizine are suspended in 150 parts of methanol. 6.4 parts of N,N-diisopropylethylamine are added and the mixture is slowly heated up and stirred at refluxed for 4 hours. After cooling down, the precipitate is separated by filtration and washed with diluted hydrochloric acid and water. Drying in an oven at 80° C. under vacuum gives rise to 11.2 parts of a blue-black solid (cpd. 1a; yield 64%).

### Example 2

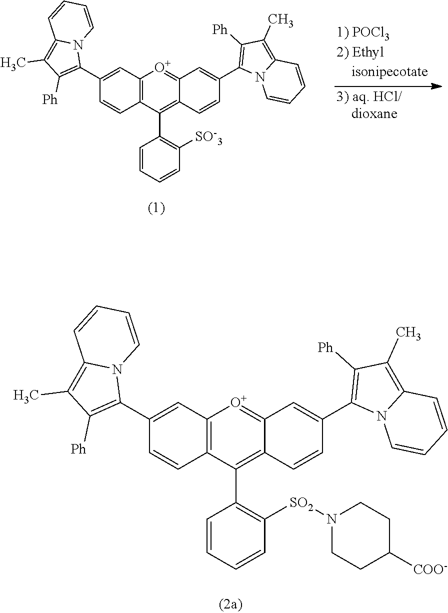

Under nitrogen atmosphere 2.4 parts of cpd. 1a is stirred with 15 parts of phosphorus(V) oxychloride for 2 hours at 90° C. The mixture is cooled down and poured onto an ice/water mixture so that the temperature doesn't exceed 5° C. The precipitate is filtered off, washed with ice cold water and dried in an oven under vacuum. The resulting sulfonic acid chloride (4) is dissolved in 200 parts of acetonitrile and cooled to 5° C.

4 parts of N,N-diisopropylethylamine and 4 parts of ethyl isonipecotate are slowly added within 10 min. After stirring for three hours the formed precipitate is filtered off and washed with 2M hydrochloric acid and water. The precipitate is added to 50 parts of a 1:1 mixture of 5M hydrochloric acid and dioxane and stirred at 90° C. for 10 hours. The precipitate is filtered off, washed with water and dried in an oven at 40° C. under vacuum. Yield: 1.2 parts of cpd. 2a as a dark powder.

1.-16. (canceled)

17. A compound of the formula: 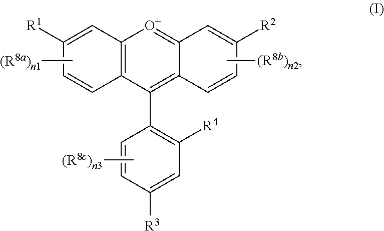
wherein
R¹ and R² are independently of each other a group of formula 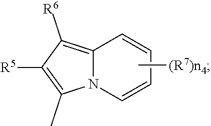
R³ is hydrogen, —CO₂H, or —SO₃H;
R⁴ is —SO₃⁻, or a group of formula —SO₂NR¹²R¹³,
wherein when R⁴ is not negatively charged, the compound of the formula (I) comprises an anionic counterion;
R⁵ is a C₁-C₂₄alkyl group, a C₃-C₁₀-cycloalkyl group, substituted with one or more substituents R¹⁴, a C₆-C₁₀aryl-C₁-C₁₀alkylene, wherein may be substituted with one or more substituents R¹⁴ and the alkylene group may be interrupted by one or more nonadjacent groups selected from 0, S and —NR⁹; a C₆-C₁₀aryl group, which may be substituted with one or more substituents R¹⁴, or a C₂-C₁₄heteroaryl group, which may be substituted with one or more substituents R¹⁴;
R⁶ is hydrogen, a C₁-C₂₄alkyl group, a C₁-C₂₄alkoxy group, a C₆-C₁₀-aryl group, which may be substituted with one or more substituents R¹⁴, and a C₁-C₂₄-fluoroalkyl group, fluorine, chlorine or bromine;
R⁷ is a C₁-C₂₄alkyl group, a C₁-C₂₄alkoxy group, a C₁-C₂₄-fluoroalkyl group, a group NR¹⁰R¹¹, fluorine, chlorine or bromine;
R^{8a}, R^{8b} and R^{8c}, independently of each other, are selected from a C₁-C₂₄alkyl group, a C₁-C₂₄alkoxy group, a C₁-C₂₄-fluoroalkyl group, fluorine, chlorine or bromine;
R⁹ is hydrogen, or a C₁-C₄alkyl group;
R¹⁰ and R¹¹ independently of each other a C₁-C₂₄alkyl group, a hydroxy-C₁-C₂₄alkyl group, a C₆-C₁₀-aryl group, which may be substituted with one or more substituents R¹⁴, or a C₃-C₁₀-cycloalkyl group, substituted with one or more substituents R¹⁴; or
R¹⁰ and R¹¹ together with the nitrogen to which they are bonded form a five, or six membered ring system, which may be interrupted by —O—, —S—, or —NR¹²′— and may be part of a fused ring system, which may be substituted with one or more substituents R¹⁴;
R¹² and R¹³ are independently of each other hydrogen, a C₁-C₂₄alkyl group, a hydroxy-C₁-C₂₄alkyl group, a C₃-C₁₀-cycloalkyl group, substituted with one or more substituents R¹⁴, a C₆-C₁₀aryl-C₁-C₁₀alkylene, wherein may be substituted with one or more substituents R¹⁴ and the alkylene group may be interrupted by one or more nonadjacent groups selected from O, S and —NR¹²′; a C₆-C₁₀aryl group, which may be substituted with one or more substituents R¹⁴, or a C₂-C₁₄heteroaryl group, which may be substituted with one or more substituents R¹⁴; or
R¹² and R¹³ together with the nitrogen to which they are bonded form a five, or six membered ring system, which may be interrupted by —O—, —S—, or —NR¹²′— and may be part of a fused ring system, which may be substituted with one or more substituents R^{14′};
R¹²′ is hydrogen, or a C₁-C₄alkyl group;
R¹⁴ is a C₁-C₁₀alkyl group, a C₁-C₁₀alkoxy group, a C₁-C₁₀fluoroalkyl group, a nitro group, a cyano group, a hydroxy group, fluorine, chlorine or bromine;
R^{14′} is a C₁-C₁₀alkyl group, a C₁-C₁₀alkoxy group, a C₁-C₁₀fluoroalkyl group, a nitro group, a cyano group, a hydroxy group, a —(CH₂)ₙ₅COOH group, a —(CH₂)ₙ₅COO⁻ group, fluorine, chlorine or bromine; and
n1 is 0, 1, or 2; n2 is 0, 1, or 2, n3 is 0, or 1, n4 is 0, 1, or 2 and n5 is 0, or an integer 1 to 10.

18. The compound according to claim 17, which is a compound of formula: 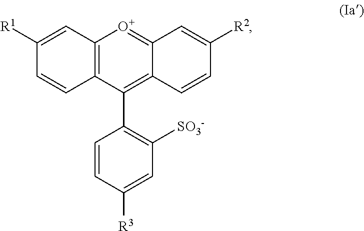
wherein R¹, R² and R³ are defined in claim 17.

19. The compound according to claim 17, which is a compound of formula: 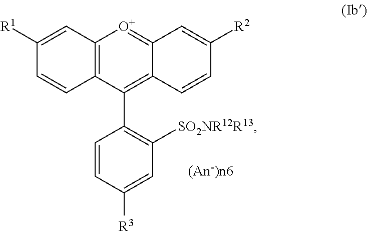
wherein n6 is 0, or 1, An⁻ is an anion and R¹, R², R³, R¹² and R¹³ are defined in claim 17.

20. The compound according to claim 19, wherein the group —NR¹²R¹³ is selected from a phenylmethylamino group, a diphenylamino group, a dimethylamino group, a methylethylamino group, a 2-hydroxyethylmethylamino group, a 2-hydroxyethylethylamino group, a di-2-hydroxyethylamino group, a 2-ethylhexylmethylamino group, a 2-ethylhexylethylamino group, a 3-propylhepthylmethylamino group and a 3-propylhepthylethylamino group; or
R¹² and R¹³ together with the nitrogen to which they are bonded form a five, or six membered ring system, which is selected from groups of formula 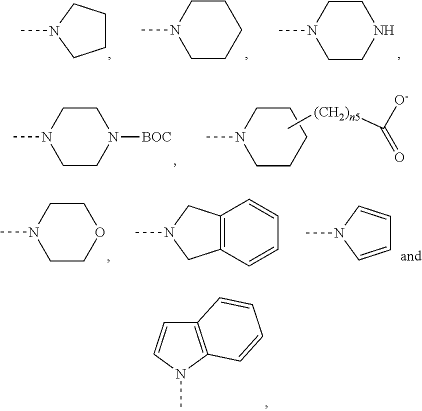
especially 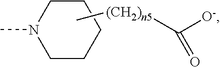
wherein BOC is tert-butyloxycarbonyl and n5 is 0, or an integer 1 to 5.

21. The compound according to claim 17, wherein
R¹ and R² are a group of formula 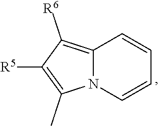
wherein R⁵ is a phenyl group, a 4-cyano phenyl group, a 4-trifluoromethylphenyl group, a 4-nitrophenyl group, a 3,5-bis-trifluoromethylphenyl group, a 4-hydroxyphenyl group, or a 4-methoxyphenyl group, especially a phenyl group; and
R⁶ is a C₁-C₄alkyl group, or a CF₃ group.

22. The compound according to claim 21, wherein R⁵ is a phenyl group and R⁶ is a methyl group.

23. The compound according to claim 17, wherein R³ is hydrogen.

24. in the compound according to claim 17, wherein the compound is configured to absorb light emitted from an irradiation source and optionally emits light different from that of the irradiation source and having a wavelength in the range from 650 to 1200 nm; or is configured to be a semiconductor in organic electronic applications; as laser dye, for bioimaging, in photodynamic therapy, in an ink for machine readability and/or security applications or for the laser-welding of plastics; or for brand protection or as marker for liquids, especially oils.

25. A printing ink formulation for security printing, comprising
a) a compound as defined in claim 17,
b) a polymeric binder,
c) a solvent,
d) optionally at least one colorant, and
e) optionally at least one further additive.

26. A security document, comprising a substrate and a compound of formula (I) as defined in claim 17, or security document, obtainable by a printing process, wherein the printing ink formulation comprises:
a) a compound of formula (I),
b) a polymeric binder,
c) a solvent,
d) optionally at least one colorant, and
e) optionally at least one further additive.

27. A method of detecting the authenticity of the security document as defined in claim 26, comprising the steps of:
a) measuring an absorbance, reflectance or transmittance spectrum of the security document in the VIS/NIR range of the electromagnetic spectrum; and
b) comparing the spectrum measured under a) and/or information derived therefrom with a corresponding spectrum and/or information of an authentic security element.

28. A composition comprising a compound as defined in claim 17 and a pharmaceutically-acceptable carrier.

29. A method for imaging a biological sample, the method comprising:
(a) contacting the biological sample with the composition of claim 28;
(b) exposing the biological sample and the composition to VIS/NIR radiation; and
(c) observing NIR fluorescence emission in the biological sample.

30. A process for the production of a compound of formula: 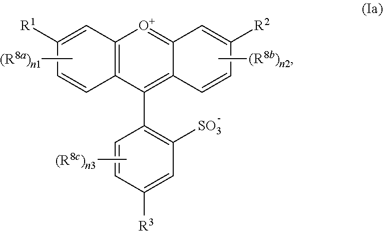
comprising:
i) reacting a compound of formula 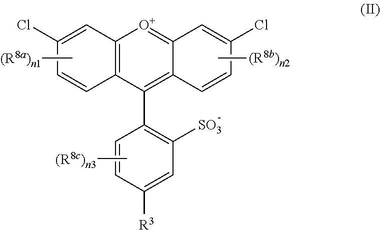
with a compound of formula 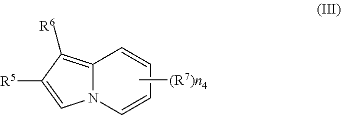
in the presence of diisopropylethylamine in a solvent, especially n-butanol, wherein n1, n2, n3, n4, R¹, R², R³, R⁵, R⁶, R⁷, R^{8a}, R^{8b} and R^{8c} are defined in claim 17.

31. The process according to claim 30, comprising
ii) converting the compound of formula 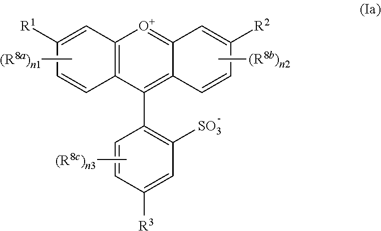
in a compound of formula 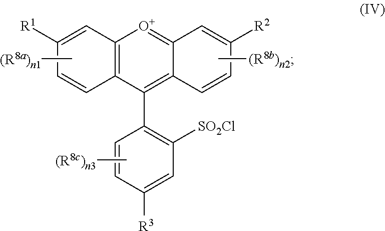
iii) reacting the compound of formula (IV) with a compound of formula HNR¹⁵—X¹—X² (V) to obtain a compound of formula 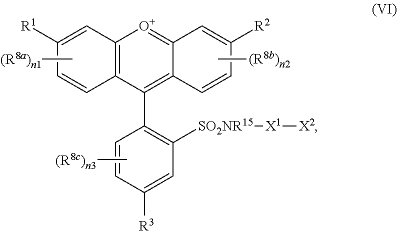
wherein X² is a reactive functional group; or reacting the compound of formula (IV) with a compound of formula HNR¹²R¹³ (VII) to obtain a compound of formula 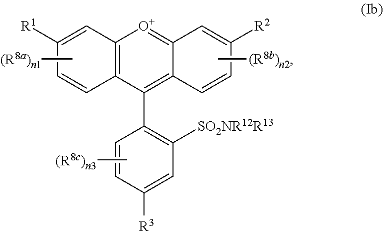
and
iv) optionally converting the compound of formula (VI) obtained in step (iii) into a compound of formula (VI), wherein
X² is a conjugated substance,
X¹ is a linking group;
R¹⁵ is hydrogen, or a C₁-C₂₄alkyl group;
R¹ and R² are independently of each other a group of formula 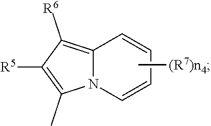
R³ is hydrogen, —CO₂H, or —SO₃H;
R^{8a}, R^{8b} and R^{8c}, independently of each other, are selected from a C₁-C₂₄alkyl group, a C₁-C₂₄alkoxy group, a C₁-C₂₄-fluoroalkyl group, fluorine, chlorine or bromine;
R¹² and R¹³ are independently of each other hydrogen, a C₁-C₂₄alkyl group, a hydroxy-C₁-C₂₄alkyl group, a C₃-C₁₀-cycloalkyl group, substituted with one or more substituents R¹⁴, a C₆-C₁₀aryl-C₁-C₁₀alkylene, wherein may be substituted with one or more substituents R¹⁴ and the alkylene group may be interrupted by one or more nonadjacent groups selected from 0, S and —NR¹²′; a C₆-C₁₀aryl group, which may be substituted with one or more substituents C₁-C₁₀alkyl group, a C₁-C₁₀alkoxy group, a C₁-C₁₀fluoroalkyl group, a nitro group, a cyano group, a hydroxy group, a —(CH₂)ₙ₅COOH group, a —(CH₂)ₙ₅COO⁻ group, fluorine, chlorine or bromine, or a C₂-C₁₄heteroaryl group, which may be substituted with one or more substituents C₁-C₁₀alkyl group, a C₁-C₁₀alkoxy group, a C₁-C₁₀fluoroalkyl group, a nitro group, a cyano group, a hydroxy group, a —(CH₂)ₙ₅COOH group, a —(CH₂)ₙ₅COO⁻ group, fluorine, chlorine or bromine; and
n1 is 0, 1, or 2; n2 is 0, 1, or 2, n3 is 0, or 1.

32. A compound of formula: 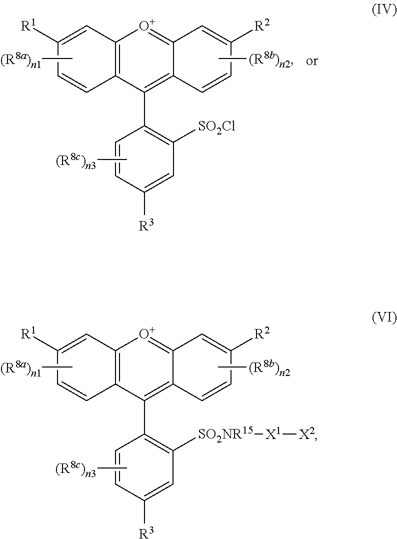
wherein
X¹ is a linking group;
X² is a reactive functional group, or a conjugated substance, and
R¹⁵ is hydrogen, or a C₁-C₄alkyl group; and
n1, n2, n3, n5, R¹, R², R³, R^{8a}, R^{8b} and R^{8c} are defined in claim 17.
